(19) United States
(12) Patent Application Publication
(10) Pub. No.: US 2013/0041308 A1
(43) Pub. Date: Feb. 14, 2013

(54) Laser Assisted Nail Avulsion

(71) Applicants: David R. Hennings, Roseville, CA (US); Elwood Phillips, Roseville, CA (US); Randolph W. Nordyke, Roseville, CA (US)

(72) Inventors: David R. Hennings, Roseville, CA (US); Elwood Phillips, Roseville, CA (US); Randolph W. Nordyke, Roseville, CA (US)

(21) Appl. No.: 13/560,343

(22) Filed: Jul. 27, 2012

Related U.S. Application Data
(60) Provisional application No. 61/512,880, filed on Jul. 28, 2011.

(51) Int. Cl. A61B 18/20; A61M 37/00

(52) U.S. Cl. 604/20; 606/9

(57) ABSTRACT

A method of treating inaccessible microbial infections, the method comprising the steps of exposing the microbe, irradiating the microbe with infrared radiation and cooling such that heat inactivates the pathogen and stimulates a wound healing response in the patient.

### RELATED APPLICATION(S)

The present application is a non-provisional application of U.S. Provisional Patent Application Ser. No. 61/512,880 filed Jul. 28, 2011 entitled “LASER ASSISTED NAIL AVULSION”, Attorney Docket No. CTI-2002-P, which is related to U.S. Ser. No. 12/841,110 filed Jul. 21, 2010, entitled “TREATMENT OF MICROBIAL INFECTIONS USING HOT AND COLD TREATMENT SHOCK AND PRESSURE”, Attorney Docket No. CTI-2001, which is a non-provisional application of U.S. Provisional Patent Application Ser. No. 61/227,739 filed Jul. 22, 2009 entitled “TREATMENT OF MICROBIAL INFECTIONS USING HOT AND COLD THERMAL SHOCK AND PRESSURE”, Attorney Docket No. CTI-2001-P, which is incorporated herein by reference in its entirety, and claims any and all benefits to which it is entitled therefrom.

### FIELD OF THE INVENTION

This invention relates to the treatment and inactivation of microbial infections. Fungal infections of the toenail are treated by removing the infected nail plate and subsequently applying a source of thermal energy to the nail bed to inactivate residues of the microbe and to improve the healing of the wound. This laser assisted therapy is more efficient, safer, and more effective than previous methods of using nail surgery or laser treatment of the nail separately.

### BACKGROUND OF THE INVENTION

As many as 700 million people worldwide suffer from onychomycosis or toenail fungal infections. There are many systemic, topical and surgical treatments available to treat this disease but none are truly efficacious and several have severe potential side effects. A need exists for a better cure for this widespread disease.

Optical and laser treatment of toenail fungus has been known for many years. In particular, UV light in the 100-400 nm range has proven to be able to inactivate many pathogens including the ones responsible for onychomycosis in non-thermal dosages. Unfortunately UV light has difficulty penetrating the toenail and can cause side effects in the dermis. UV light is not considered to be a successful treatment modality despite a great deal of research.

U.S. Pat. No. 6,723,090, issued Apr. 20, 2004 to Altshuler et al., U.S. Pat. No. 7,220,254, issued May 22, 2007 to Altshuler et al., US Publication No. 2006/0212098, published Sep. 21, 2006 to Demetriou et al., Non-patent publication “Laser treatment for toenail fungus”, Proc. of SPIE Vol. 7161 published 2009 by Harris et al. and others have proposed using infrared radiation to thermally inactivate toenail fungus. Infrared radiation penetrates the toenail much better than UV and it has been shown that the fungus can be inactivated by raising the temperature of the pathogen to about 50 oC. The problem associated with this method is that achieving the inactivation temperature in the nail bed risks damaging the surrounding dermal tissue, especially the matrix where the nail actually grows. In addition this prior art allows the use of infrared radiation with high hemoglobin absorption. Hemoglobin absorbing wavelengths can coagulate capillaries in the proximal fold and permanently damage the toenail.

U.S. Pat. No. 6,723,090, issued Apr. 20, 2004 to Altshuler et al., U.S. Pat. No. 7,220,254, issued May 22, 2007 to Altshuler et al. propose to use a cooling modality to protect the toenail during infrared laser irradiation to target the nail bed and he suggests that a pulsed laser may be superior to a continuous one.

US Publication No. 2006/0212098, published Sep. 21, 2006 to Demetriou et al. suggests the use of pulsed cryogen cooling, which is also described in U.S. Pat. No. 5,814,040, issued Sep. 29, 1998 to Nelson et al., to protect the toe from excessive heating and to use the process of selective photothermolysis, which is disclosed in non-patent publication “Selective Photothermolysis: Precise Microsurgery by Selective Absorption of Pulsed Radiation”, published on Science, 220:524-527, 1983 by Anderson et al., to choose the correct pulse length to match the thermal properties of the fungus itself Methods taught respectively in U.S. Pat. No. '090, '254 to Altshuler et al. and US Publication '098 by Demetriou et al. all require relatively high target temperatures that can damage the matrix and teach to cool only the surrounding tissue. The above-mentioned methods may cause permanent damage to sensitive areas.

U.S. Pat. No. 6,090,788, issued Jul. 18, 2000 to Lurie teaches that light-absorbing substances may be considered to induce and enhance selective photothermal damage. The problem and shortcoming with this method is the difficulty in getting the substance infused to the proper areas and the high temperatures required to inactivate the microbe. Damage to the surrounding tissue is likely to happen by using this method.

Capon and others have published non patent descriptions of the wound healing and scar reduction capabilities of thermal laser energy when applied to a surgical site immediately pre and post op. The mechanism of action of this wound healing effect is postulated to be the activation of a heat shock protein.

Nail removal or avulsion has been used to treat badly infected toenails but without success. The fungal infection is not completely eliminated even with the removal of the nail and the area usually becomes re infected even with the application of topical antifungal medications and good foot hygiene. It is not obvious that laser treatment after nail avulsion would be of any benefit. Only after months of experimentation with laser power settings did the Inventors discover that there is a level of laser energy and method of application that simultaneously provides hemostasis, microbe inactivation, and wound healing. Laser energy at too low a power setting is ineffective and the fungus returns. Laser power at too high a setting will ablate the tissue and permanently injure the patient or kill the nail matrix and prevent re growth of the nail plate.

Chato has shown that heat alone is effective in inactivating fungus if it can be held at 50 deg C. for about 5 min It is almost impossible to do this in vivo as surrounding tissue will be destroyed along with the fungus. Others have tried to heat the fungus through the nail with limited success. One device made by PinPointe, Inc. is able to only show a partial clearing of the nail after several laser treatments through the nail. This device is applied through the nail and is done without any anesthesia preventing therapeutic temperature levels from being achieved safely. This device also does not monitor surface temperature so the precise therapeutic temperature cannot be safely achieved. Clinical results with this device have shown only a 20% reduction in the infected area of the nail at 12 months post op.

### ADVANTAGES AND SUMMARY OF THE INVENTION

The present invention makes the treatment of inaccessible microbial infections more effective and efficient than previously taught in the prior art.

The goal of the present invention is to improve efficacy of treatment of nail disease. Nail Avulsion alone is about 50% effective and laser treatment of nail bed promotes collagen formation and wound healing. Combined treatment is more effective than avulsion alone.

This invention is composed of the following steps:
a. Anaesthetizing the toenail with a local anesthesia.
b. Removing the overlying nail plate of the toenail to expose the infection.
c. Irradiating the open wound with sufficient energy to cauterize broken capillaries.
d. Irradiating the open wound to desiccate the infection, reducing microbe activity.
e. Irradiating the exposed fungal infection to thermal energies capable of inactivating the microbes.
f. Thermally injuring the exposed tissue to stimulate a wound healing effect but with energy low enough to preserve the nail matrix.

The present invention utilizes a thermal feedback sensor and laser controller to precisely control the energy delivered to the site. Without a thermal feedback mechanism it is not possible to maintain a treatment surface temperature that will cauterize, inactivate, desiccate, and stimulate, while preserving the ability to re grow a healthy new nail.

The present invention utilizes a laser that is only partially absorbed in the target tissue. Laser energy that is absorbed too shallow will not penetrate deep enough to stimulate a wound healing effect. Laser energy that penetrates too deeply will not heat the surface tissue hot enough to inactivate the microbes and may damage underlying structure including the matrix area that generates new nail growth. The present invention uses a laser wavelength that is absorbed in water which assists in desiccation of the infections and provides better hemostasis of ruptured capillaries.

The present invention utilizes a cooling mechanism that will cool surrounding tissue and prevent collateral damage during laser irradiation.

The present invention adds highly absorbing dyes and/or metallic nanoparticles such as gold nanoparticles GNP to enhance the absorption by the targeted fungus. Furthermore, the use of GNP causes photothermal micro-bubbles PTMB at the surface of the GNP, which in turn provide an effective way of promoting non-thermal mechanical and localized inactivation of microbes. Prior art has not taught the use of GNP to inactivate living microbes. Prior art has only utilized GNP to physically ablate nonliving tissue such as plaque.

The present invention adds a photo active compound to the treatment site to accelerate the inactivation of the microbe. This compound can be Riboflavin or vitamin B, which will absorb UV light and become active to kill the fungus in the treatment area.

The present invention adds a topical antifungal that is delivered directly to the treatment site in conjunction with laser delivery and whose effect is enhanced by the thermal heating properties of the laser.

Thus, it is an object of the present invention to make the treatment of inaccessible microbial infections much more effective and efficient.

It is yet a further object of the present invention to provide treatment of microbial infections using laser energy transmitted via fiber optic laser delivery device.

It is yet a further object of the present invention to provide an improved method and apparatus for treatment of microbial infections of toenails, including onychomycosis.

### BRIEF DESCRIPTION OF THE DRAWINGS

FIG. 1 is a representative illustration showing an embodiment of the apparatus and method of treatment of microbial infections using thermal inactivation and wound healing of exposed infections of the present invention 100.

FIG. 2 is a representative illustration showing an embodiment of a transparent toe jacket 200 of the present invention 100.

FIG. 3 is a representative illustration showing an embodiment of laser control system with cooling spray device devices and methods of the present invention 100.

FIG. 4 is a representative illustration of laser assisted wound healing.

FIG. 5 is a representative illustration of laser assisted nail avulsion.

### DETAILED DESCRIPTION OF THE PREFERRED EMBODIMENT

The description that follows is presented to enable one skilled in the art to make and use the present invention, and is provided in the context of a particular application and its requirements. Various modifications to the disclosed embodiments will be apparent to those skilled in the art, and the general principals discussed below may be applied to other embodiments and applications without departing from the scope and spirit of the invention. Therefore, the invention is not intended to be limited to the embodiments disclosed, but the invention is to be given the largest possible scope which is consistent with the principals and features described herein.

It will be understood that in the event parts of different embodiments have similar functions or uses, they may have been given similar or identical reference numerals and descriptions. It will be understood that such duplication of reference numerals is intended solely for efficiency and ease of understanding the present invention, and are not to be construed as limiting in any way, or as implying that the various embodiments themselves are identical.

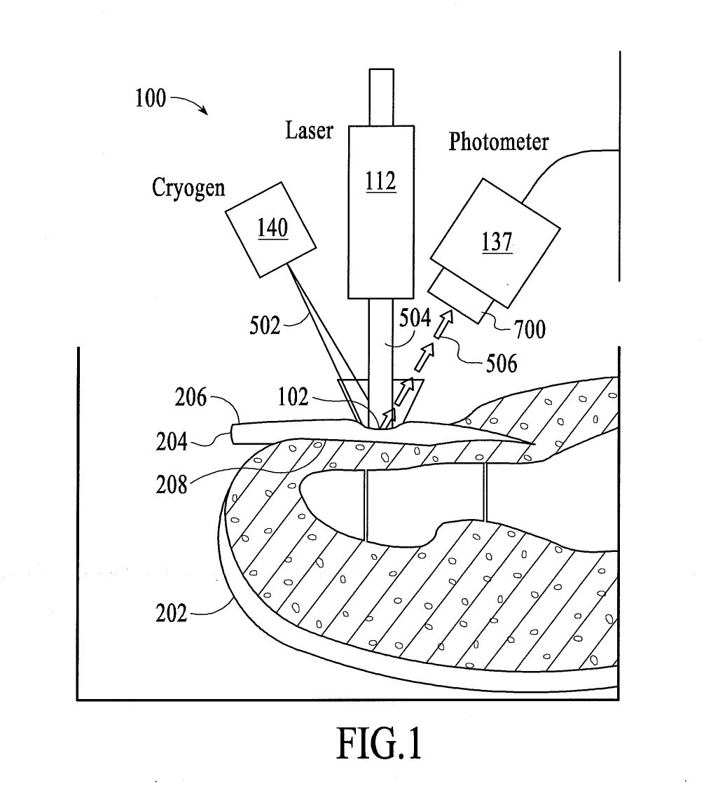

FIG. 1 is a representative illustration showing an embodiment of the apparatus and method of treatment of microbial infections using thermal inactivation and wound healing of the present invention 100.

The present invention 100 uses an automatic target thermal feedback to precisely control the dosimetry of the laser 112, or intense light or intense pulsed light IPL irradiation, to prevent damage to surrounding tissue and reduce pain. A non-contact thermal detector 137, such as made by Raytek or equivalent, is built into a handpiece along with a lens to focus the laser delivery fiber optic 504 or a laser diode. The output of the non-contact thermal detector 137 is used to adjust the power output of the laser 112 to maintain a selected treatment temperature at the treatment site 102.

A preferred embodiment of the present invention utilizes a 1320 nm continuous or pulsed laser 112 that is capable of delivering 2 to 5 watts of energy, or more or less, with continuous or pulsed cryogen cooling 140. The energy is delivered from a handpiece that focuses the light into a 2-10 mm diameter spot on the treatment tissue, treatment site 102. A non contact thermal sensor 137 detects the temperature of the treated spot and send a signal to the laser 112 control system which then adjusts the energy to maintain a pre selected target temperature at the spot. A continuous or pulsed cooling spray device 140 is incorporated into the handpiece to deliver a spray of coolant 502 to the target treatment spot 102 after each laser treatment interval.

It will be understood that the site of infection is associated with the nail 204 of the finger or toe 202 the nail has a plate 206 as well as a bed 208.

Preferential absorption of laser energy 290 having a wavelength of 1320 nm to 1470 nm by the nail bed 206 of the infected toe or finger 204 results in a controlled elevation in temperature to a temperature effective at disinfection of the infected regions or areas without causing irreversible thermal damage to the infected nails.

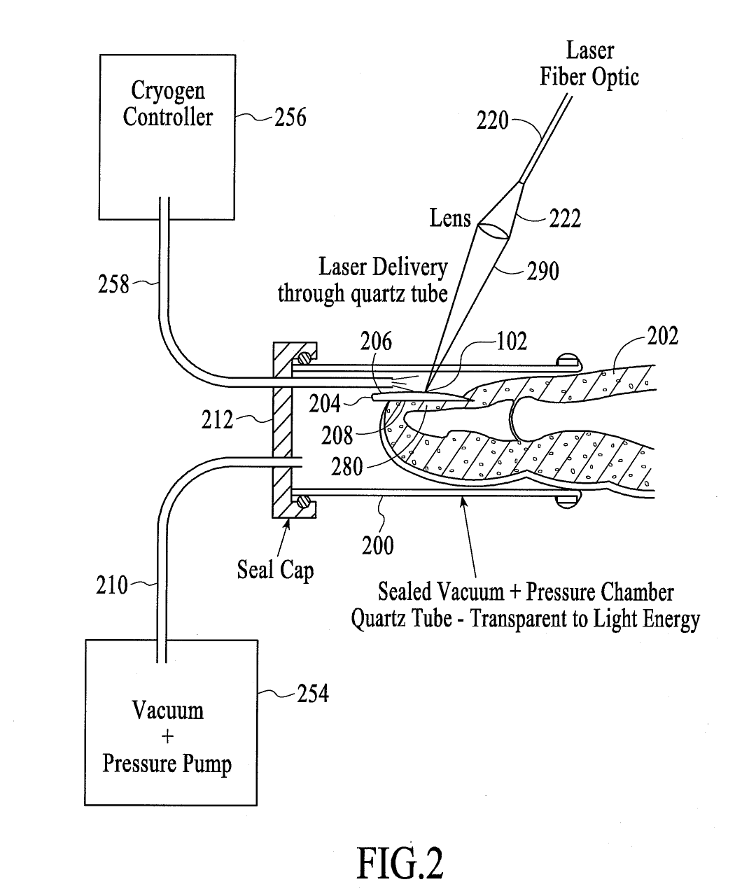

FIG. 2 is a representative illustration showing an embodiment of a transparent toe jacket 200 of the present invention 100. As best shown in FIG. 2, fiber optic laser delivery system 220 comprises optical fibers as well as lens mechanism 222, and optional filters, convertors or other beam modifiers which can be coupled to the toe 200 as desired.

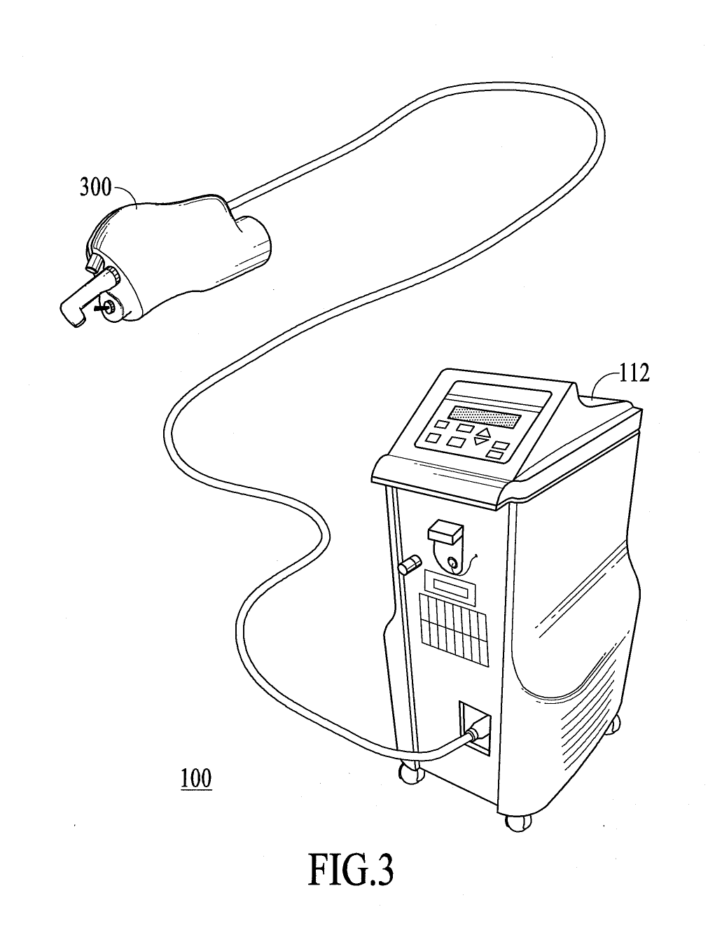

FIG. 3 is a representative illustration showing an embodiment of laser control system with cooling spray device devices and methods of the present invention 100. As described above, a preferred embodiment of the present invention utilizes a 1320 nm continuous or pulsed laser 112 that is capable of delivering 2 to 5 watts of energy, with continuous or pulsed cryogen cooling. The energy is delivered from a handpiece 300 that focuses the light into a 2-10 mm diameter spot on the target treatment spot 102. The laser 112 control system adjusts the energy to maintain a pre selected target temperature at the spot. A continuous or pulsed cooling spray device is incorporated into the handpiece 300 to deliver a spray of coolant to the target treatment spot 102 after each laser treatment interval.

The laser and coolant delivery handpiece 300 can be the CoolTouch® TQ10 model handpiece or equivalent. In an embodiment, the handpiece 300 can deliver laser energy 290 at a wavelength of 1320 nanometers at a fluence rate of 24 Joules per square centimeter. The handpiece 300 with integrated continuous or pulsed cryogen cooling reduces the surface temperature for protection allowing the laser energy 290 to be effectively targeted. Cooling can be provided adjustably cooling to maximize patient comfort, safety and efficacy.

### Treatment Agents

It will be understood that THE PRESENT INVENTION consists of applying liquid or gas directly to the target, i.e., to the infected nail. Furthermore, the liquid or gas may contain one of more of the following: pain reducing agent, antifungal agent, anti-microbial agent, antiseptic agent or disinfectant agent. It will be understood that there are a wide range of agents which are associated with pain reduction, anti-irritant, antifungal treatment, antimicrobial and antibiotic activity as well as antiseptic and disinfecting properties, the use of which is expressly contemplated herein.

Antifungal agents may include any antifungal agents useful in dermatological compositions. Examples of antifungal agents include, without limitation, Tea Tree oil and other naturally occurring oils and compounds, nystatin, ciclopirox and ciclopirox olamine, griseofulvin, itraconazole, fluconazole, ketoconazole, terbinafine, econazole, benzyl alcohol, undecylenic acid and salts thereof, benzyl benzoate and combinations thereof. Antifungal agents are well known in the art. One of ordinary skill in the art would understand, appreciate and recognize agents that are considered to be antifungal agents.

Antimicrobial agents may include any antimicrobial agents useful in dermatological compositions. Antimicrobial agents include, without limitation, benzoyl peroxide, povidone iodine, hexachlorphene, chlorhexidine, mupirocin, gentimycin, neomycin, bacitracin, polymixin, erythromycin, clindamycin, metronidazole, clarithromycin, silver sulfadiazine, dapsone, zinc pyrithione, cephalosporin, thymol, mafenide acetate, nitrofurazone, generators of nitric oxide benzyl alcohol, sulfamethoxazole, sulfasalazine, sulfasoxazole, acetylsulfasoxazole and combinations thereof. Antimicrobial agents are well known in the art. One of ordinary skill in the art would understand, appreciate and recognize agents that are considered to be antimicrobial agents.

Anti-irritants are well known in the art. One of ordinary skill in the art would understand, appreciate and recognize agents that are considered to be anti-irritants. Preferred anti-irritants include but are not limited to aloe vera gel, alpha bisabolol, allantoin, sorbitol, urea, lactic acid and salts, glucose derivatives, zinc acetate, zinc carbonate, zinc oxide, potassium gluconate, dimethicone, glycerin, petrolatum, lanolin, peramides, uric acid and salts, N-acetyl cysteine, and hydrocortisone.

Disinfectants are also well known in the art. One of ordinary skill in the art would understand, appreciate and recognize agents that are considered to be disinfectants. Preferred disinfectants include but are not limited to chlorine bleach or sodium hypochlorite

Antiperspirants are used post op to control moisture in the wound area. Preferred antiperspirants include over the counter under arm sprays such as.

### Method Of Treatment

The following is taken from the CoolBreeze® (trademark) treatment guidelines for onychomycosis.

### Patient Preparation:

1. Remove all lotions and skin care products, making certain that the skin of the foot and nail bed are completely dry prior to treatment.
2. Local anesthetics are recommended for the CoolBreeze mode. The local anesthetic is administered by injection to each infected toe. Anesthetics allow therapeutic energy levels to be used with patient comfort.
3. Remove the infected nail using standard avulsion techniques. Scrape away any infected residue under the nail.

Setting Treatment Parameters:

Fluence: Set at 1-5 watts for the CoolTouch 1320 nm laser.

Adjust as needed to achieve effective hemostasis and tissue heating.

When using higher fluences, the nail bed will reach target temperature more quickly and the speed of hand piece movement will need to be faster.

Target Temperature: Set at 39° C. (Range is 30° C.-42° C.)

The system will sound an audible alert, “Beep” when the target nail bed temperature is reached, as well as displaying the temperature on the control panel.

Each subsequent pass will increase temperature and the target temperature may be reached more quickly than anticipated.

Cryogen Cooling: Set at 30 msec (Range is 0-50 msec)

Cryogen will be delivered after the target temperature has been achieved.

NOTE: These guidelines are meant to establish starting parameters. In any given clinical procedure there are many variables involved, therefore the settings may need to be modified to accomplish the desired treatment goals.

The CoolBreeze® Mode

Micro-pulses of laser energy are delivered continuously when the foot pedal is depressed.

When the target nail bed temperature is reached, system will emit an audible high pitched, rapidly repeating, “beep”. And the firing of the laser will slow.

Target temperature is displayed continuously on the display panel.

Movement of the hand piece

The speed of the hand piece movement and the selected fluence should allow the patient to experience mild to moderate warmth but not a sensation of hot or pain.

Target temperature and the confirming audible beep will be reached quicker with each additional pass.

Lightly glide the gold footplate just above the tissue surface, avoiding treatment to the surrounding skin overlap by manipulating the hand piece in a smooth continuous motion.

Keep the hand piece perpendicular to the nail surface.

Each pass may be changed to a different orientation of movement for a more uniform distribution of energy.

Multiple passes will be needed before moving to the next toe.

Suggested Treatment Interval: Every month for a total of 3-4 treatments

Treat the nail as it grows out with laser energy and topical anti fungal medications to re stimulate the wound healing response The number of the treatments is based on the condition of the nail and the amount of improvement desired.

Toenails will re grow in a few months and the improvement should be seen immediately.

Post Procedure Care:

Wear comfortable shoes and hosiery that allow your feet some breathing space.

Wear shoes, sandals or flip-flops in community showers or locker rooms.

Wash your feet every day, dry them thoroughly and use an antiperspirant. Ask your doctor to recommend an antiperspirant with the right blend of ingredients.

Wear clean socks or stockings every day.

Keep toenails trimmed.

Disinfect pedicure tools before and after you use them. Note: Be sure to wipe the footplate with an appropriate disinfectant when finish treating each patient and before storing the handpiece.

Experimental Results

The present invention comprises the step of exposing the infected tissue by removal of the nail plate and irradiating the nail bed with infrared radiation using laser energy having a wavelength between about 800 nm and about 2000 nm, and more particularly, using laser energy having a wavelength of about 1200 to 1600 nm. A preferred wavelength is 1320 nm

The present invention further comprises the step of exposing the infected tissue by removal of the nail plate and irradiating the nail bed with infrared radiation using laser energy having a wavelength between about 1450 nm and about 1550 nm, and more particularly, using laser energy having a wavelength of about 1470 nm.

Experiment I:

The CoolTouch® 1320 nm laser was used to treat infected toes in 50 individuals with a single avulsion treatment and follow up laser treatments every 5 weeks. New clear growth was seen at three months and the nails are completely clear at 6 months post treatment.

Background:

Organisms that cause onychomycosis can invade both the nail bed and the nail plate. Dermatophytoses of the fingernails and toenails, in contrast to those at other body sites, are particularly difficult to eradicate with drug treatment. This is the consequence of factors intrinsic to the nail—the hard, protective nail plate, sequestration of pathogens between the nail bed and plate, and slow growth of the nail, as well as of the relatively poor efficacy of the early pharmacologic agents.

The efficacy of current treatment options, including topical, oral, mechanical and chemical therapies or a combination of these modalities is low. Topical drug treatment for onychomycosis is not usually successful because they are unable to penetrate the nail plate, as disclosed in Crawford, F, Young P, Godfrey C et al., “Oral treatments for toenail onychomycosis: a systematic review,” Arch Dermatol 138, 811-816 (2002) and Elewski, B. E., “A full ‘cure’ for onychomycosis is not always possible,” Arch Dermatol 135, 852-853 (1999), and rapid recurrence can occur after discontinuing use. Oral antifungal agents are more effective although more toxic with a significant risk of liver toxicity, prolonged loss of taste, and life-threatening drug interactions as discussed in Katz, H. I., “Drug interactions of the newer oral antifungal agents,” Br J Dermatol 141(Suppl. 56), 26-32 (1999). Fungal resistance can occur when the oral antifungal agents are used on a long-term basis. Topically applied antifungal drugs may work somewhat better adjunctive to surgical removal or chemical dissolution of the nail plate as disclosed in Grover, C., Bansal S., Nanda S. et al., “Combination of surgical avulsion and topical therapy for single nail onychomycosis: a randomized controlled trial,” Br I Dermatol 157, 364-368 (2007). But the results were still poor and this study concluded that “Surgical nail avulsion followed by topical antifungal therapy cannot be generally recommended for the treatment of onychomycosis.” This current invention adds the critical new step of laser irradiation immediately post operative and improves the results of nail avulsion dramatically.

Device Description:

The CoolTouch® CT3P CoolBreeze 1320 nm 18W pulsed Nd:YAG laser is an FDA (K043046) cleared device and is indicated for use in dermatology for incision, excision, ablation and vaporization with hemostasis of soft tissue.

The unique handpiece design of the CoolTouch® laser allows the operator to maintain a constant distance from the area to be treated resulting in constant and uniform energy delivery. Treatment spot size is adjustable from 3 mm to 10 mm allowing pre-selection of the optimal spot size for the nail being treated. The energy delivered to the toenail can be adjusted by the selecting the desired level of watts (1.5 W to 12 W) with a push of a single control panel key. The CT3P CoolBreeze™ laser has a unique thermal sensing mechanism design to control the amount of energy delivered to the toenail by pre-setting the desired end target temperature. In addition, patient comfort is assured by a spray of a cooling agent when the target temperature is reached. Unlike other laser systems, having the fiber enclosed and terminated in the handpiece means that the fiber does not need cleaving during or after the laser procedure.

In this early assessment of the CoolTouch® CT3P CoolBreeze 1320 nm laser for the treatment of onychomycosis, no attempt was made to narrow the cohort of patients by selective eliminating those patients with proximal infections and nail matrix involvement, the very difficult to treat patient group usually non-responsive to pharmacologic agents. A two or three laser treatment regimen allows much higher patient compliance with the treatment protocol, very high patient safety with minimal side effects. Documented high patient satisfaction with minimal patient reported pain or discomfort suggests a safe and tolerable procedure. In addition, using the CoolTouch® CT3P CoolBreeze laser the procedure can be performed in less than 15 minutes (total treatment time for both feet and all toes with multiple passes) and allows effective utilization of valuable physician time.

Improved nail clearing demonstrated with these preliminary results support the hypotheses that the 1320 nm wavelength, using controlled energy delivery and a cooling spray is an effective treatment modality that inhibits or destroys the dermatophyte pathogens that cause onychomycosis resulting in high patient satisfaction.

Concurrently-owned U.S. Pat. No. 5,820,626 entitled COOLING LASER HANDPIECE WITH REFILLABLE COOLANT RESERVOIR, U.S. Pat. No. 5,976,123 entitled HEAT STABILIZATION, U.S. Pat. No. 6,451,007 entitled THERMAL QUENCHING OF TISSUE, U.S. Pat. No. 7,122,029 entitled THERMAL QUENCHING OF TISSUE, U.S. Pat. No. 6,413,253 entitled SUBSURFACE HEATING OF MATERIAL, are hereby incorporated herein in their entireties in regards to their teaching of methods and apparatus for cryogenic cooling as part of an overall medical, dermatological and/or aesthetic treatment.

### EXAMPLE II

### Of Treatment Protocol

CoolBreeze® Treatment Guideline for Laser Assisted Nail Avulsion

Laser assisted nail avulsion is a new and novel technique that provides the physician with a successful methodology to provide clear nails for the most difficult to treat subset of patients with onychomycosis. The cohort for this treatment is the approximate 20% of patients who do not respond well to the use of simple laser treatment to produce clearing of the nail. This difficult to treat group include those patients with decades long standing disease, all ten nails infected, highly dystrophic, yellowed nails and are usually elderly.

The CoolBreeze® laser produces 1320 nm laser energy that is specifically heats the tissue of the nail bed and inhibits dermatophyte growth and re-infection of the tissues. In addition, this heating of the nail bed tissue after nail removal coagulates the surface of the nail bed tissues and stimulates a wound healing response.

CoolBreeze® lasers provide a continuous delivery of energy, controlled by selecting the desired target temperature, that results in heating a larger volume of tissue and penetrating to a deeper depth. This provides an effective treatment just below the pain threshold, heating the nail bed to produce the desired anti-fungal, wound healing and nail rejuvenation effect.

The following guidelines are meant to establish starting parameters. In any given clinical procedure there are many variables involved, therefore the settings may need to be modified to accomplish the desired treatment goals. As with any medical procedure the final responsibility and treatment choice lies with the practitioner.

Patient Selection and Preparation:
Nail avulsion is a surgical procedure with a significant associated recovery time. Proper patient selection is critical.
These patients should be selected from that group that has responded poorly or not at all to standard laser treatments for onychomycosis.
Perform a digital block for anesthesia to toes that are selected for nail avulsion.
Rubber banding is recommended to use as a tourniquet to restrict venous flow and provide a bloodless field.
Remove nail using a standard surgical technique.
It is important to scrap nail bed and remove all debris from nail folds.
Provide laser safety eyewear for operator and patient.

Setting Laser Treatment Parameters: (Post Nail Avulsion):
I. Set spot size to 3 mm
II. Set power to 3.0 Watts
III. Set cryogen to 10 msec
IV. Set target temperature to 38° C.

1. Starting at 3 Watts, treat the avulsed area using a continuous motion and multiple passes until the entire nail bed is at target temperature (this may take 10 or more cryogen sprays.) The nail bed should appear coagulated, semi-dry with minimal exudate. It is very important to treat matrix and lateral/medial folds.

2. Increase power to 3.5 Watts and treat a second time.

3. The third and final pass is at 4.0 Watts for all avulsed nail beds.

Note: These guidelines are meant to establish starting parameters. In any given clinical procedure there are many variables involved, therefore the settings may need to be modified to accomplish the desired treatment goals. As with any medical procedure the treatment choice and final responsibility lies with the practitioner.

Treatment Technique Hints:

Note: Before each procedure inspect handpiece lens for contamination and clean the lens if necessary. Refer to Operation Manual for detailed instructions.

The CoolBreeze® Mode:
Micro-pulses of laser energy are delivered continuously when the foot pedal is depressed.
When the target nail bed temperature is reached, system will emit an audible high pitched, rapidly repeating, “beep”. The firing of the laser will slow and cryogen will be sprayed on to the nail plate.
Target temperature is displayed continuously on the display panel.
The red aiming beam spot indicates the approximate size of the treatment area. However, the mid-infrared treatment beam overlaps the red spot and is invisible to the naked eye.

Movement of the Hand Piece:
Target temperature and the confirming audible beep will be reached quicker with each additional pass and over areas of the avulsed nail..
Lightly glide the gold footplate across the nail surface, avoiding treatment to the proximal and lateral folds
Move the hand piece in a smooth and continuous motion.
To maintain an accurate temperature reading, do not stop movement of the handpiece during the laser treatment.
Keep the hand piece spacer tip contacting the nail and perpendicular to the nail surface.
Each pass may be changed to a different orientation of movement for a more uniform distribution of energy.
Multiple passes (reaching target temperature with cryogen cooling spray each time) will be needed before treating the next toe.

Treatment Interval:
Reschedule patient for a 2nd treatment four (4) weeks from initial treatment date. Treat with laser as outlined above.
Since toenails grow very slowly, improvement is not seen immediately. Changes in the nail bed are cellular in nature and take time Improvement may be seen over a period of several months as the undamaged nail grows out.

Post Procedure Care:
Post avulsion—use gauze 4×4's and then wrap with appropriate elastic bandaging materials.
Post-op regimen: topical antibiotic—Neosporin b.i.d. (30 days), anti-perspirant starting immediately post op.
Dry avulsed nail beds, re-bandage after application of Neosporin.
Wear comfortable shoes and hosiery that allow your feet adequate breathing space.
Wear shoes, sandals or flip-flops in community showers or locker rooms.
Wear clean socks or stockings every day.

Note: Be sure to wipe the gold treatment tip with an appropriate disinfectant after treating each patient and before storing the hand piece.

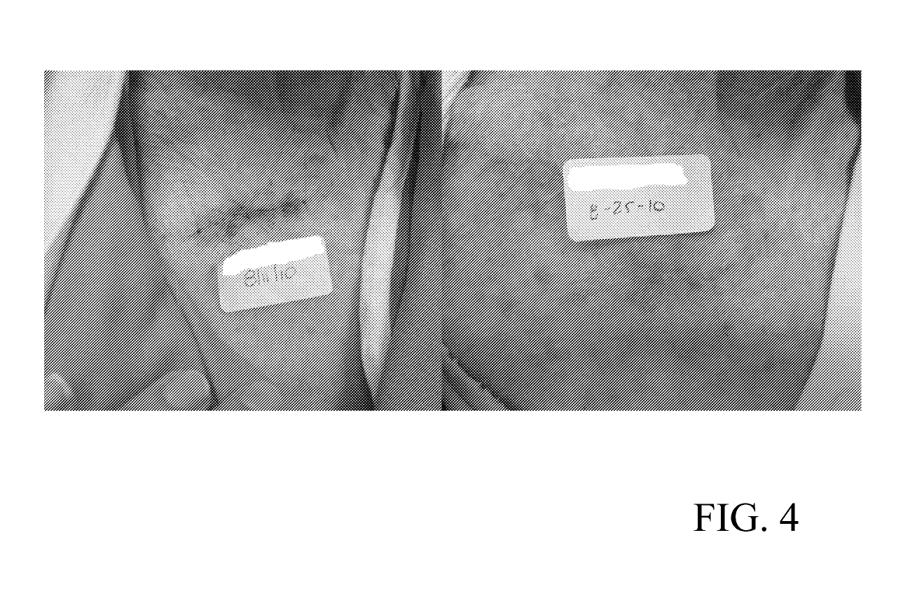

FIG. 4 is a representative illustration of laser assisted wound healing. As shown in FIG. 4, CoolBreeze® 1320 laser assisted wound healing of R. G. Geronemus et al., clinical study in 2011.

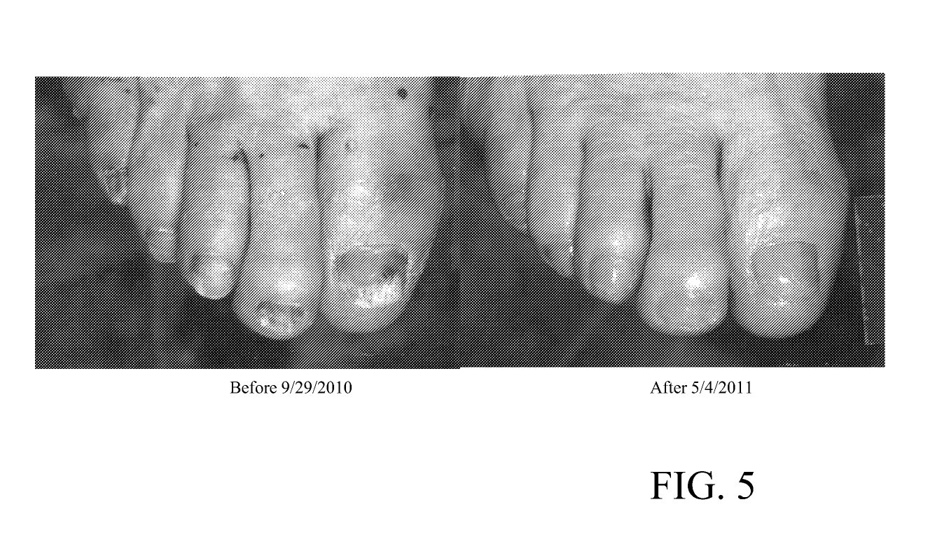

FIG. 5 is a representative illustration of laser assisted nail avulsion. As shown in FIG. 5, CoolBreeze® 1320 laser assisted nail avulsion in R. Nordyke clinical study in January 2011.

Unless defined otherwise, all technical and scientific terms used herein have the same meaning as commonly understood by one of ordinary skill in the art to which the present invention belongs. Although any methods and materials similar or equivalent to those described can be used in the practice or testing of the present invention, the preferred methods and materials are now described. All publications and patent documents referenced in the present invention are incorporated herein by reference.

While the principles of the invention have been made clear in illustrative embodiments, there will be immediately obvious to those skilled in the art many modifications of structure, arrangement, proportions, the elements, materials, and components used in the practice of the invention, and otherwise, which are particularly adapted to specific environments and operative requirements without departing from those principles. The appended claims are intended to cover and embrace any and all such modifications, with the limits only of the true purview, spirit and scope of the invention.

We claim:

1. A method of treating inaccessible microbial infections, the method comprising the steps of exposing the microbe, irradiating the microbe with infrared radiation and cooling such that heat inactivates the pathogen and stimulates a wound healing response in the patient.

2. The method of claim 1, further comprising the step of using non-contact thermal measurement of the infected site to automatically control either or both the heating and the cooling of the site.

3. The method of claim 1, further comprising the step of using a laser in the wavelength range of 800 nm to 1800 nm.

4. The method of claim 1, further comprising the step of treating the site of infection with GNP or other particle seeds to create a rapid and violent thermoelastic expansion or cavitation of the tissue surrounding the microbial infection during infrared radiation exposure, thereby improving treatment efficiency.

5. The method of claim 1 in which the site of the infection is the human toe or finger nail wherein the method further comprises the step of irradiating the human toe or finger nail, thereby treating the toe or finger nail fungus.

6. The method of claim 5, further comprising the step of using temporary modification of the optical properties of the toe skin, thereby reducing or increasing the incidence of back scattering/absorption.

7. The method of claim 1, wherein the step of irradiating the microbe with infrared radiation using energy from a filament lamp or a pulsed arc lamp.

8. The method of claim 1 wherein the step of irradiating the microbe with infrared radiation is performed using laser energy having a wavelength between about 1200 nm and about 2000 nm

9. The method of claim 1 wherein the step of irradiating the microbe with infrared radiation is performed using laser energy having a wavelength of about 1320 nm.

10. The method of claim 1 wherein the step of irradiating the microbe with infrared radiation is performed using laser energy having a wavelength between about 1450 nm and about 1550 nm

11. The method of claim 1 wherein the step of irradiating the microbe with infrared radiation is performed using laser energy having a wavelength of about 1470 nm.

12. The method of claim 1 further comprising the step of cooling with a liquid or gas applied directly to the target.

13. The method of claim 1 further comprising the step of applying liquid or gas directly to the target, the liquid or gas containing one of more of the following: pain reducing agent, antifungal agent, anti-irritant agent, antimicrobial agent, antibiotic agent, antiseptic agent, disinfectant agent.

14. A method of laser assisted nail avulsion that combines thermal inactivation of the microbe with photo dynamic control of the microbe.

15. The method of claim 15 where the photodynamic material is Riboflavin or Vitamin B

16. The method of claim 15, further comprising the step of first irradiating the microbe with infrared radiation followed by irradiating the microbe with Ultraviolet radiation in a range that is absorbed by the photodynamic agent.

17. The method of claim 1, further including the step of using pulsed radiation with a wavelength that is selectively absorbed by the microbes and with a pulse length that is chosen to match the thermal diffusion properties of the microbe.

18. The method of claim 1, further comprising the use of a wavelength that is absorbed by water to desiccate the tissue area.

19. The method of claim 1, further comprising the step of introducing GNP or other particle seeds to create a rapid and violent thermoelastic expansion or cavitation of the microbial infection, thereby increasing the disinfection efficiency during laser irradiation.

20. The method of claim 1 that involves the treatment of toenail fungus or infections.

21. The method of claim 20 that uses the temporary modification of the optical properties of the toe skin to either reduce or promote back scattering/absorption.

22. The method of claim 20 that uses optical clearing agents to allow energy to penetrate deeper into selected parts of the nail bed.

23. The method of claim 20 that involves the use of glycerin, or DMSO prior to laser irradiation.

24. The method of claim 16 wherein the step of irradiating the microbe with infrared radiation is performed using laser energy having a wavelength between about 1450 and about 1550 nm.

25. The method of claim 16 wherein the step of irradiating the microbe with infrared radiation is performed using laser energy having a wavelength of about 1470 nm.
